(19) United States
(12) Patent Application Publication
(10) Pub. No.: US 2008/0085248 A1
(43) Pub. Date: Apr. 10, 2008

(54) Controlled Long Acting Release Pharmaceutical Preparation For Use In The Oral Cavity

(76) Inventor: Yoram Sela, Raanana (IL)

Correspondence Address: Hershkovitz & Associates, LLC, 2845 Duke Street, Alexandria, VA, 22314, US

(21) Appl. No.: 11/794,168

(22) PCT Filed: Dec. 28, 2005
(86) PCT No.: PCT/IL05/01385

§ 371 Date: Jun. 26, 2007

(30) Foreign Application Priority Data
Jan. 3, 2005 (IL) 166114

(51) Int. Cl. A61K 8/21; A61K 8/36

(52) U.S. Cl. 424/49; 424/52

(57) ABSTRACT

The present invention relates to a controlled long acting release pharmaceutical preparation for use in the oral cavity either for the treatment of diseases of the oral cavity or; for releasing said pharmaceutical preparation into said oral cavity. Said pharmaceutical preparation comprises at least a therapeutic agent, a binder and a lubricant (hereinafter “the preparation” or “the pharmaceutical preparation”).

### CROSS-REFERENCE TO RELATED APPLICATIONS

This disclosure claims priority under 35 U.S.C. § 119 of Israel Patent Application No. 166,114, filed on Jan. 3, 2005, and under 35 U.S.C. § 371 of International Patent Application No. PCT/IL2005/001385, filed on Dec. 28, 2005.

### BACKGROUND OF THE INVENTION

1. Field of the Invention

This invention relates to a controlled long acting release pharmaceutical preparation for use in the oral cavity e.g. in the treatment of dental caries, treating local diseases of the oral cavity, delivery of drugs with preferred absorption at the upper parts of the GI tract, drugs intended for chronic use, etc. The invention relates also to a method for retaining and delivering the above controlled long acting release pharmaceutical preparations in the oral cavity.

The present invention is described herein with reference to the treatment of dental caries but is not restricted thereto.

2. Discussion of Background Information

Dental caries is a disease in which minerals of the tooth are dissolved into surrounding bacterial plaques and to the saliva. Dental caries has a multi factorial etiology in which there is interplay of three principal factors: the host (saliva), the micro flora (plaque), and the substrate (diet). A fourth factor is time. The mechanism of caries formation is well described in the literature.

Tooth decay has plagued humans for centuries and, although its causes are fairly well understood, an efficient method for the prevention of teeth decay doesn't exist.

Teeth are protected by a hard enamel layer, about 2 mm thick. The dental enamel is a crystalline latticework composed of various minerals. The principal component of enamel is a complex of calcium phosphate called Hydroxy Apatite Ca(PO₄)₃OH. (Ca₅(PO—O₃OH). Teeth demineralization and re-mineralization processes occur all the time in the oral cavity. Removal of mineral ions is a key factor of the demineralization process. When sugar and other fermentable carbohydrates reach the bacteria which are a permanent part of the mouth's natural flora, the bacteria form acids which start to dissolve the enamel. An early caries lesion occurs, due to loss of calcium and phosphate ions. The demineralization process has a severely detrimental impact on the strength and hardness of the dental enamel. The reverse process, re-mineralization, is the body's natural defense against the tooth decay.

Normally after a meal, bacteria in the mouth break down food to produce organic acids such as acetic and lactic acids. This acid formation causes a local decrease in the oral cavity's pH, resulting in the removal of OH— ions and initiating the demineralization process. The dental enamel mineral is practically a “living stone” and, as elsewhere in nature, acids dissolve the enamel minerals, transforming them from solid mineral molecules into mineral ions which exist only in solution. Strong acids are stable and very small quantities are sufficient to continue dissolving the enamel minerals. By the removal of billions of calcium and other mineral ions from the Hydroxyapatite latticework, the enamel loses its structural integrity and tooth decay starts.

In the re-mineralization process, the body utilizes carbon dioxide from breathing, and water from the saliva to create Carbonic Acid. The Carbonic Acid dissolves minerals in the saliva (minerals originating from food). As the Carbonic Acid reverts to Carbon Dioxide and water, the mineral ions precipitate out as a solid mineral ion and may be deposited onto a demineralized portion of the Hydroxyapatite crystal and incorporated into the enamel lattice.

Although this process is always occurring, its level of activity is strongly dependent on the conditions within the oral cavity. At least six conditions have been identified as being necessarily present. Sufficient minerals have to be present in the saliva, a function of diet, correct chewing and sufficient salivation. Carbonic Acid molecules must be produced (salivation and breathing) and the Carbonic Acid molecules must be produced in proximity to mineral molecules which then dissolve into its ionic components. This all has to occur in proximity to a demineralized spot in the Hydroxyl Apatite lattice that requires mineral ions. The demineralized portion of the enamel has to be clearly accessible to allow the mineral ions to be attracted to the hole in the lattice by an opposite electric charge. Finally, Carbonic Acid molecules must be converted back to Carbon Dioxide and water. When all this happens, the mineral ion precipitates out of the solution into the structure of the enamel.

In our modern life, demineralization is enormously accelerated by the quantity of refined sugars and processed foods in our diet, and cannot be balanced by natural remineralization alone.

Calcium is an essential component in many of the basic body functions; bone metabolism, tooth development, nerve transmission, etc. About 1% percent of the body's supply of Calcium goes into the formation and maintenance of bone and teeth. Since it is constantly shuttled from the bones to be utilized in other bodily needs, its maintenance is crucial. Hormones and vitamin D control the body's use of Calcium. The skeleton and teeth contain 99% of the total body Calcium in a crystalline form resembling the mineral Hydroxylapatite (Caio(PO₄)₆(OH₂), but other ions including: Na+, K−, F, Mg2+ are also present in a crystal lattice. The steady state content of Calcium in the bone reflects the net effect of bone formation and resorption. In addition to its basic role in re-mineralization processes, Calcium and Phosphate ions, which are normally present in the mouth, have certain buffer capacity.

The recommended dietary allowance of Ca for adolescents and adults up to the age of 24 years is 1200 mg/day and for older adults 800 mg/day. Ca₂ enters the body only through the intestine by two different mechanisms: 1) active, Vitamin D dependent transport in the proximal duodenum, and 2) diffusion through the small intestine.

Current avenues for attempting to deal with dental caries include: preventive efforts such as: increasing saliva secretion (chewing gum containing Calcium Fluoride, etc.), mouthwash preparation with Fluoride and sterilizing agents; tooth pastes containing Fluoride and other additives, (for example a tooth paste is now known which paste comprises Sodium Fluoride plus Calcium and Phosphate ions); and in development stages are strategies employing genetic engineering to prevent adhesion of bacteria to the teeth surface, and/or to prevent the acidic secretion on teeth.

Additionally, Calcium ions, in spite of their known crucial role on bone and tooth re-mineralization processes are supplied as immediate release (“IR”) preparations like: IR tablets, tooth pastes and dental flosses.

Long acting products are widely marketed in the pharmaceutical field and are now a significant factor in the administration of a variety of active pharmaceutical agents.

The advantages of long acting, or sustained release products, are now well understood and a very substantial industry has developed around these products.

Furthermore, numerous drugs administrated per-os are absorbed efficiently only in the upper gastrointestinal tract, namely, the stomach and the proximal section of the small intestine. The passage of drugs from the stomach to the intestine is normally too fast (usually, between one or two hours), strongly limiting the bioavailability of these drugs. Since the residence time of drug at the site of optimal absorption largely determines its bioavailability, it is apparent that prolonging the retention of the drug-containing device in the proximal gastrointestinal tract is of the utmost importance. There are important features that have to be considered when the treatment of a certain disease has to be decided. Delivery of a drug at a constant rate from the oral device may assist in maintaining a constant level of the released drug and to overcome the blood and tissue variable concentration due to diurnal variation in the intake of the drug by the patients. A controlled long acting release pharmaceutical preparation may ease medical treatment and improve patient's compliance. Said pharmaceutical preparation may also be used for the local treatment of a variety of oral cavity diseases, like: Candiadisis, Fungal, etc.

The prior art provided controlled release dosage forms that can administer a drug continuously over time for controlled rate therapy as in for example, U.S. Pat. No. 4,327,725 and in U.S. Pats. Nos. 4,612,008, 4,765,989, and 4,783,337. The dosage forms disclosed in these patents provide a controlled rate drug delivery over an extended time to provide less erratic drug therapy, and eliminating the need for multiple dosing of drug. These dosage forms can deliver many drugs for their intended therapy, but there are certain drugs that are not readily manufactured and delivered in CR. dosage forms. For example, controlled release Calcium preparations alone or combined with therapeutic adjuvants.

U.S. Pat. Nos. 5,049,077 and 5,137,449 (Golding) disclose various orthodontic appliances adapted for holding intra-oral Fluoride releasing tablets. However, the patents do not teach a method which can promote re-mineralization or which will address the difficulties of delivering Calcium in sufficient quantities and over a long term.

### SUMMARY OF THE INVENTION

It is apparent in the light of the above presentation that an urgent need exists for a dosage form endowed with controlled release delivery for the administration of pharmaceutical preparations to the oral cavity. The need exists in particular for dosage forms for delivering Calcium ions as a preventive therapy against the formation of dental caries, and to enhance re-mineralization processes in the oral cavity. The need is particularly acute with respect to patients using dental devices (braces, dental bridges, etc.) who are more susceptible to formation of dental caries, for integration as part of routine orthodontic treatment.

In general, an oral device such as a device for long-term pharmaceutical application such as drug release can significantly improve treatments with drugs that are taken for long periods, as is the case of chronic diseases, hormonal treatments, as well as simplify treatments that combine several different drugs.

It is in particular an object of the present invention to provide long-acting release pharmaceutical preparations for the treatment in the oral cavity, in particular calcium-containing preparations which release Calcium and other adjuvant additives e.g. Zinc, Phosphate, Casein Phosphopeptide, Fluoride and other components, in a controlled manner into the oral cavity, in order to enhance the re-mineralization processes, buffer the salivary pH, and minimize the acidic unwanted effects of the plaque activity. The exemplary Calcium-containing preparations are controlled release dosage forms, e.g. tablets, pills, etc., provided in sizes and shapes suitable for installation and retention in the oral cavity. Preferably, the preparations and the retention methods and devices should be well tolerated by the patient for periods of weeks at a time.

In a desire to be applicable to both orthodontic patients as well as users having no oral appliances, it is highly desirable to provide a slow release dosage form which will either be retained by itself or will be held in place by a device fitted into the mouth.

Such a device must be as non-intrusive as possible and yet still capable of retaining the oral dosage form for a period of days, weeks, or even months. Furthermore, such a device must be able to receive new replacement dosage forms. Additionally, as much of the dosage form surface should be exposed to the saliva to promote distribution of the active ingredients as widely and efficiently as possible.

### DETAILED DESCRIPTION OF THE PRESENT INVENTION

The present invention thus includes a controlled long acting release pharmaceutical preparation for use in the oral cavity either for the treatment of diseases of the oral cavity or for releasing said pharmaceutical preparation into said oral cavity, comprising at least a therapeutic agent, a binder and a lubricant (hereinafter “the preparation” or “the pharmaceutical preparation”).

The present invention also includes a drug based on any suitable pharmacological active materials. In said preparation said diseases are advantageously human or veterinary diseases. Moreover, said preparation is compatible with many types of dental designs.

The present invention also includes the above preparation comprising further a rate controlling polymer as a carrier, a filler, a glidant, a rate controlling polymer as a coating material, alone or with pore forming agents.

The therapeutic agent according to the preparation of the present invention is dependant on the treatment of the specific disease to be treated by the specific preparation. Said agent should be: e.g. a) For Dental Caries:

Calcium Salts or Calcium Salts in Combination with Their Therapeutic Adjuvants.

Such Calcium salts may be Calcium Fluoride, Calcium Hydroxide, Calcium Oxide, Calcium Saccharate, Calcium Sulphate, Calcium Acetate, Calcium Chloride, Calcium Citrate, Calcium Glubionate, Calcium Gluceptate, Calcium Gluconate, Calcium Lactate, Calcium Lactate Gluconate, Calcium Lactobionate, Calcium Laevulinate, Calcium Hydrogen Phosphate, Calcium Pidolate, Calcium Sodium Lactate, in particular:

Calcium Carbonate, Calcium Phosphate and Calcium Glycerophosphate.

Such therapeutic adjuvants may be Casein, Phosphopeptide, Zinc etc., b) For Anticandidiasis, Antifungal Treatments: Nystatin, Imidazols, Ciclopirox, Clotrimazole etc. c) For Antiseptic Treatment d) Breath refreshers used in the treatment of Xerostomv e) Treatment of mouth ulcerations such as: Aphthous Stomatitis/Cancer Sores/oral herpetic infections (HSV): Triamcinolone Acetonide, Tetracycline, etc. f) Anasthetics: Lidocaine; and g) Drugs with preferred absorption at the upper parts of the GI tract: Immunosuppressants: such as Sirolimus, Tacrolimus, etc.; Antivirals: such as Acyclovir, Anti HIV agents, etc.; Azidothimidine (AZT); Antiparkinsonian: such as Levodopa and Carbidopa, etc.; and Antibiotics: such as Ciprofloxacin, etc.

The pharmaceutical preparation according to the present invention may also comprise Klucel EF from 5% to 25%, Calcium glycerophosphate from 75% to 95% and from 0.2% to 1% lubricant, wherein the release mechanism is based on diffusion.

The pharmaceutical preparation according to the present invention may also comprise Ethyl cellulose from 5% to 25%, calcium glycerophosphate from 75% to 95%, and from 0.2% to 1% lubricant wherein the release mechanism is based on erosion.

The binder according to the preparation of the present invention may be any suitable binder, e.g. PVP, Methocel, Ethocel (Ethylcellulose), Klucel, etc.

The lubricant according to the preparation of the present invention may be any suitable lubricant, e.g. Syloid, PEG, Mg Stearate, Pruv etc.

In a further embodiment of the present invention the preparation may be provided with an additional suitable functional/rate controlling coating layer being any suitable rate controlling polymer such as: Ethocel (Ethylcellulose), HPMC (Hydroxypropyl Methylcellulose), Cellulose Acetate, Acrylic polymers, Polyox, (high MW polyethylene oxide), etc.

Polymers which may also optionally be used as carrier base materials in controlled release matrix based tablets, pills, pellets, gels or capsules include, e.g. Hydrophilic polymers including Hydroxypropylmethyl Cellulose (HPMC) and Hydroxypropyl Cellulose (HPC); GUMS, Polyethylene Oxide (Polyox) and Polyvinyl Pyrolidone (PVP); Hydrophobic polymers such as Ethyl Cellulose (Ethocel); Acrylate based polymers such as Eudragit, PVA, Povidone and functional mixtures such as Kollidone SR.

Two options to control the release of the active compound: first by polymeric coatings; second polymers (many times the same as used in polymer coating) may be used as matrix components in which upon hydration, hydrogels which control the release are formed.

In a further embodiment the coating layer is a semi permeable membrane, and the dosage form comprises osmotic components.

Additional excipients which may be part of the preparation according to the present invention may include, e.g.; Fillers, such as Microcrystalline Cellulose, Lactose, etc., and flavors, and other acceptable tablets forming components, e.g. flavors, may include Menthol, Saccharin, Vanillin, etc.

The preparation according to the present invention for use in the oral cavity advantageously comprises a therapeutic agent in the amount of from 0.1 mg to 1000 mg, a binder in the amount of from 1% w/w to 10% w/w of the preparation and a lubricant in the amount of from 1% w/w to 10% w/w of the preparation.

Said preparation comprises preferably the following components:

A therapeutic agent in the amount of from 0.1 mg to 1000 mg, from 1% w/w to 10% w/w of the preparation of a binder, from 5% w/w to 50% w/w of the preparation of a rate controlling polymer, from 1% w/w to 10% w/w of the preparation of a glidant, from 1% w/w to 10% w/w of the preparation used as a lubricant and a pore forming agent used in amount from 5% w/w to 50% w/w of the dry polymer.

The pharmaceutical preparation according to the present invention may be used, e.g. for preventing dental caries, treating local diseases of the oral cavity, delivery of drugs with preferred absorption at the upper parts of the GI tract, for drugs intended for chronic use etc.

The pharmaceutical preparation according to the present invention is compatible with many types of dental/orthodontic devices and with most types of oral dosage forms.

The preparation may be provided in unit dosage form, such as an oral tablet, which insures that the unit dosage form has uniform and comparable in vitro and bioavailability characteristics. This object of the invention is achieved by providing a carrier base material comprising at least one polymeric material, such as Cellulose Ethers, Acrylic Polymers and other therapeutically suitable polymers for oral administration, but not limited to polymer carriers, which polymeric material is thoroughly mixed with Calcium Salts, alone or combined with therapeutic adjuvants, to form pharmaceutical preparations. Other controlled release technology which may be appropriate comprises use of special pore-forming coatings, which allow controlled release of the active ingredients through pores in the coating in an osmotic manner or any known method and which permits controlled release of the therapeutic agents.

The oral cavity is thus buffered, less acidic, thereby minimizing the damage caused by acidic secretion of the oral bacteria to the teeth. Current known preparations containing Calcium Phosphate, Fluoride, (like mouth wash preparations) can shift the balance to re-mineralization only for a very short period.

The preparations to be used may be intended for the treatment of pathological situations in the oral cavity, for preparations intended for chronic use, and with preferred absorption at the upper part of the GI tract-stomach and to a small intestine.

The present invention comprises also a method for the preparation of the pharmaceutical preparation as defined above and for the use thereof.

The dosage forms of the pharmaceutical preparations may be prepared e.g. in accordance with the coated preparations or with the semi-permeable membrane technologies disclosed in U.S. Pat. Nos. 4,340,054; 4,450,198; 4,008,719; 4,519,801, the contents of which are incorporated herein in their entirety.

The present invention comprises a method for applying long term anti-caries treatment based, e.g. on controlled slow release of Calcium and Phosphate ions to the oral cavity, in particular to dental caries in orthodontic patients.

The present invention comprises preparations particularly well-suited for long term intra-oral deposition and mineral or drug release in the singular environment of the oral cavity.

The present invention comprises also a method for promoting tooth re-mineralization and for applying a long term anti-demineralization therapy.

Such a device should be in particular suitable for maintaining a mineral, organic or combined dosage form in position in the oral cavity for a period of time which may range from several hours to a month or more.

The devices according to the present invention may be attached to the teeth in the form of mechanical devices, of holders, or of suitably bio compatible or bio-absorbable adhesive. As an example, customized holders, containing the controlled release dosage forms, may be an integral part of the therapy which will be part of the orthodontic treatment.

Such devices may be devices as described in U.S. Pat. Nos. 4,485,805, 4,681,544 and 4,741,700.

The present invention also includes the use of a pharmaceutical preparation according to the present invention, being stored in a device located in the oral cavity either for the treatment of diseases of the oral cavity or for releasing the pharmaceutical preparation into said oral cavity.

The controlled release of the preparation according to the present invention may be achieved by storage of the preparations in the interior of the oral cavity, where it is dissolved by the saliva, which becomes a carrier for the preparation. For this purpose it is necessary to install, store, and retain the preparation inside the oral cavity. Moreover, the preparation has to be placed in the interior of the mouth when depleted. One has also to take into consideration the nature of the preparation and the CR properties. This problem is solved in the form of a device coupled for example to at least one tooth. At least one tooth may support a retaining device, which in turn, retains the preparation, or may support a housing having an interior volume for containing the preparation therein, or a housing with the preparation therein may be directly and releasably coupled to at least one tooth. The preparation may be replaced on the retaining device or by replacing the housing.

The present invention comprises also preparing a slow release dosage form of active moieties intended to treat local pathologic conditions in the oral cavity. It also improves absorption of drugs with preferred absorption at the upper parts of the GI tract. Such slow controlled release dosage forms may be in a tablet, a capsule, a pellet, a film, a pill or any other suitable oral dosage form.

In order to achieve the unmet need of gastric retention different approaches have been tested, with no proven success.

The advantages of the pharmaceutical preparations according to the present invention are further described below (in particular for the treatment of dental caries).

Controlled release in the oral cavity without any bioadhesion to the oral tissues (less tissue irritation). It may last for weeks, like no other similar treatments where only few hours are considered. Thus for anticaries treatment, the replacement can be from once weekly up to one or two months. For other potential applications, from a few hours to once monthly, require simple and safe control release (CR) preparations, and there is no need for adhesion enhancers, penetration enhancers, etc. In general the buccal tissues remain intact.

The present invention also comprises a device for maintaining a controlled long acting release pharmaceutical preparation dosage form in position in the oral cavity for a period of time which may range from several hours to a month or more.

The present invention will now be described with reference to the accompanying examples without being limited by them.

### EXAMPLES OF PREPARATIONS FOR THE TREATMENT OF SPECIFIC DISEASES

### Examples Related to Dental Caries

In general the preparation may be prepared by one of the following methods:

1. Calcium salts and: binders, lubricating agents, directly compressed into tablets.

2. Calcium salts with binders, release rate controlling polymers, lubricating agents, directly compressed into tablets

3. Like 1, 2 but instead of direct compression, wet granulation is used.

4. Like 1, 2, 3 with additional functional/rate controlling coating layer.

5. Like 1, 2, 3, 4 when the coating layer is semi permeable membrane, and the dosage form together with osmotic components like NaCl acts as Osmotic Pump.

### Example 1

| | | | |
|---|---|---|---|
| Calcium Hydroxide | 100 mg | 56%- | Therapeutic agent |
| Zinc | 5 mg | 3%- | Adjunct additive |
| Casein Phosphopeptide | 10 mg | 6% | Adjunct additive |
| Hydroxypropyl | 30 mg | 17% | Rate controlling polymer |
| Methylcellulose | | | |
| Syloid 244 | 4 mg | 2% | Glidant |
| Magnesium Stearate | 2 mg | 1% | Lubricant |
| Cellulose Acetate | 15 mg | 8% | Coating agent |
| Polyethylene Glycol | 2 mg | 1% | Pore forming agent(coating) |
| Lactose | 10 mg | 6% | Filler |

### Example 2

| | | | |
|---|---|---|---|
| Calcium Sulphate | 100 mg | 56%- | Therapeutic agent |
| Zinc | 5 mg | 3%- | Adjunct additive |
| Casein Phosphopeptide | 10 mg | 6% | Adjunct additive |
| Poly Ethyleneoxide | 30 mg | 17% | Rate controlling polymer |
| Syloid 244 | 4 mg | 2% | Glidant |
| Magnesium Stearate | 2 mg | 1% | Lubricant |
| Cellulose Acetate | 15 mg | 8% | Coating agent |
| Polyethylene Glycol | 2 mg | 1% | Pore forming agent(coating) |
| Lactose | 10 mg | 6% | Filler |

### Example 3

Calcium salt amount of 50-1500 mg.

Binder like PVP, Methocel, etc. from 1% w/w to 10% w/w of the dosage form weight.

Rate controlling polymers (Ethocel, HPMC, Polyox, etc.) from 5% w/w to 50% w/w of the dosage form.

Lubricants Syloid, PEG, Mg Stearate from 1% w/w to 5% w/w of the dosage form.

Coating (Eugragit, CA, Ethocel, etc.) from 5% w/w to 20% w/w of the dosage form.

Osmotic Agents Salts like NaCl, Sugars, etc. From 1% w/w to 5% w/w of the Dosage Form.

### Example 4

Calcium Glycerophosphate: from 50 mg to 1500 mg.

PVP K-30 from 10 mg to 100 mg.

Syloid 244 from 5 mg to 50 mg.

Mg Stearate from 5 mg to 50 mg.

The process—direct compression, blending and compression of all preparation components.

### Example 5

Calcium Carbonate: from 50 mg to 1500 mg. PVP K-30 from 10 mg to 100 mg.

Syloid 244 from 5 mg to 50 mg.

Mg Stearate from 5 mg to 50 mg.

Casein Phosphopeptide from 50 mg to 1000 mg.

The process—direct compression, blending and compression of all preparation components.

### Example 6

Calcium Carbonate from 50 mg to 1500 mg.

Calcium Phosphate from 50 mg to 1500 mg.

PVP K-30 from 10 mg to 100 mg.

Syloid 244 from 5 mg to 50 mg.

Mg Stearate from 5 mg to 50 mg.

The process—direct compression, blending and compression of all preparation components.

### Example 7

Calcium Carbonate from 50 mg to 1500 mg.

Calcium Phosphate from 50 mg to 1000 mg.

Casein Phosphopeptide from 50 mg to 1000 mg.

PVP K-30 from 10 mg to 100 mg.

Syloid 244 from 5 mg to 50 mg.

Mg Stearate from 5 mg to 50 mg.

The process—direct compression, blending and compression of all preparation components.

### Example 8

Calcium Phosphate from 50 mg to 1000 mg. Casein Phosphopeptide from 50 mg to 1000 mg. PVP K-30 from 10 mg to 100 mg. Syloid 244 from 5 mg to 50 mg. Mg Stearate from 5 mg to 50 mg.

The process—direct compression, blending and compression of all preparation components.

### Example 9

Calcium Phosphate from 50 mg to 1000 mg.

PVP K-30 from 10 mg to 100 mg.

Syloid 244 from 5 mg to 50 mg.

Mg Stearate from 5 mg to 50 mg.

The process—direct compression, blending and compression of all preparation components.

### Example 10

Calcium Carbonate from 50 mg to 1500 mg.

Calcium Phosphate from 50 mg to 1000 mg.

PVP K-30 from 10 mg to 100 mg.

Syloid 244 from 5 mg to 50 mg.

Mg Stearate from 5 mg to 50 mg.

The process—wet granulation with water or ethanol.

### Example 11

Calcium Carbonate from 50 mg to 1500 mg.

Calcium Phosphate from 50 mg to 1000 mg.

Casein Phosphopeptide from 50 mg to 1000 mg.

PVP K-30 from 10 mg to 100 mg.

Syloid 244 from 5 mg to 50 mg.

Mg Stearate from 5 mg to 50 mg.

The process—Wet granulation with water or Ethanol.

Anticandidiasis, Antifungal Drugs.

General preparation:

Nystatin, Imidazole, Ciclopirox, Clotrimazole.

### Example 12

Nystatin from 100,000 units to 2,000,000 units. Binder 10 mg.

Rate controlling polymers 20 mg Lubricants 5 mg.

### Example 13

Clotrimazole from 5 mg to 100 mg.

Binder 5 mg.

Rate controlling polymers 25 mg.

Lubricants 5 mg.

Administration—overnight to once weekly.

Treatment of mouth ulcerations (Aphthous Stomatitis/Cancer Sores) Anasthetics like Lidocaine, Triamcinolone Acetonide, Tetracycline

### Example 14

Tetracycline from 200 mg to 1000 mg. Binder 50 mg

Rate controlling polymers 250 mg Lubricants 30 mg

### Example 15

Lidocaine from 1 mg to 50 mg. Binder 3 mg

Rate controlling polymers 20 mg

Lubricants 4 mg

Administration—overnight to once weekly.

Drugs with preferred absorption at the upper parts of the oral cavity

The preparation is prepared for example as follows: Immunosuppressant, Antiparkinsonian, Antiviral Binder from 1% w/w to 10% w/w of the dosage form. Rate controlling polymers from 5% w/w to 50% w/w of the dosage form. Lubricants—from 1% w/w to 10% w/w of the dosage form.

### Example 16

Other Immunosuppressant

Tacrolimus from 1 mg to 50 mg.

PVP from 1% w/w to 10% w/w of the dosage form.

Ethocel 45 cp from 5% w/w to 50% w/w of the dosage form.

Syloid 244 from 1% w/w to 10% w/w of the dosage form.

Mg Stearate from 1% w/w to 5% w/w of the dosage form.

Once monthly preparation.

### Example 17

Levodopa 200 mg. Carbidopa 50 mg. HPMC K15M 100 mg. PVP K3010 mg. Lactose Anhydrous 50 mg. Once daily dosage form.

### Example 18

Acyclovir 500 mg.

Ethyl Cellulose 45 cp 20 mg.

Microcrystalline Cellulose 100 mg.

Klucel HF 15 mg.

Mg Stearate 5 mg.

Once weekly dosage form.

1. A controlled long acting release pharmaceutical preparation for the oral cavity either for the treatment of diseases of the oral cavity or for releasing said pharmaceutical preparation into said oral cavity, comprising at least a therapeutic agent, a binder and a lubricant.

2. The pharmaceutical preparation of claim 1, in the form of a drug comprising suitable pharmacological active materials.

3. The pharmaceutical preparation of claim 1, wherein the diseases are at least one of (1) a human disease or human diseases and (2) veterinary disease or veterinary diseases.

4. The pharmaceutical preparation of claim 1, wherein said pharmaceutical preparation is compatible with dental devices.

5. The pharmaceutical preparation of claim 1, comprising a therapeutic agent in the amount of from 0.1 mg to 1000 mg, a binder in the amount of from 1% w/w to 10% w/w of the preparation and a lubricant in the amount of from 1% w/w to 10% w/w of the preparation.

6. The pharmaceutical preparation of claim 1, further comprising a rate controlling polymer as a carrier, a filler, a glidant, and a rate controlling polymer as a coating material.

7. The pharmaceutical preparation of claim 6, which comprises a therapeutic agent in the amount of from 0.1 mg to 1000 mg, from 1% w/w to 10% w/w of the preparation of a binder, from 5% w/w to 50% w/w of the preparation of a rate controlling polymer, from 1% w/w to 10% w/w of the preparation of a glidant, from 1% w/w to 10% w/w of the preparation used as a lubricant and a pore forming agent used in amount of from 5% w/w to 50% w/w of the dry polymer.

8. A method of treating dental caries, comprising treating dental caries utilizing the pharmaceutical preparation of claim 1.

9. The method of claim 8, wherein the therapeutic agent is a Calcium salt or a Calcium salt in combination with therapeutic adjuvants.

10. The method of claim 9, wherein the therapeutic agent is at least one agent selected from the group consisting of Calcium Carbonate, Calcium Phosphate and Calcium Glycerophosphate.

11. The method of claim 9, wherein the Calcium salts are at least one salt selected from the group consisting of Calcium Fluoride, Calcium Hydroxide, Calcium Oxide, Calcium Saccharate, Calcium Sulphate, Calcium Acetate, Calcium Chloride, Calcium Citrate, Calcium Glubionate, Calcium Gluceptate, Calcium Gluconate, Calcium Lactate, Calcium Lactate Gluconate, Calcium Lactobionate, Calcium Laevulinate, Calcium Hydrogen Phosphate, Calcium Pidolate and Calcium Sodium Lactate.

12. The method of claim 9, wherein the therapeutic agent is at least one adjuvant selected from the group consisting of Casein Phosphopeptide and Zinc.

13. The pharmaceutical preparation of claim 1, further comprising 5% to 25% Klucel EF, 75% to 95% calcium glycerophosphate and 0.2% to 1% lubricant, wherein the release mechanism is based on diffusion.

14. The pharmaceutical preparation of claim 1, further comprising 5% to 25% Ethyl cellulose, 75% to 95% calcium glycerophosphate, and 0.2% to 1% lubricant wherein the release mechanism is based on erosion.

15. The pharmaceutical preparation of claim 1, wherein the therapeutic agent is utilized for anticandidiasis and antifungal treatments and is at least one member selected from the group consisting of Nystatin, Imidazols, Ciclopirox and Clotimazole.

16. The pharmaceutical preparation of claim 1, wherein said therapeutic agent is used for antiseptic treatment.

17. The pharmaceutical preparation of claim 1, wherein said therapeutic agent is used for breath refreshers used for the treatment of Xerostomy.

18. The pharmaceutical preparation of claim 1, wherein the therapeutic agent is used for the treatment of mouth ulcerations such as Aphthous Stomatitis/Cancer Sores selected among Triamcinolone Acetonide, and Tetracycline.

19. The pharmaceutical preparation of claim 1, wherein the therapeutic agent is selected from the group consisting of an anesthetic and Lidocaine.

20. The pharmaceutical preparation of claim 1, having a preferred absorption at the upper parts of the GI tract.

21. The pharmaceutical preparation of claim 20, wherein said pharmaceutical preparation is at least one member selected from the group consisting of:
any pharmacologically active material which is released at the upper parts of the G.I. tract, Immunosuppressants, Sirolimus, Tacrolimus, Antivirals, Acyclovir, Anti HIV agents, Azidothimidine (AZT), Antiparkinsonian agents, Levodopa, Carbidopa, Antibiotics, and Ciprofloxacin.

22. The pharmaceutical preparation of claim 1, said preparation being a capsule, a film, a pellet, a tablet, a pill, a gel or any other acceptable solid pharmaceutical dosage form or a combination thereof used in oral delivery of pharmaceutical preparations.

23. The pharmaceutical preparation of claim 1, wherein the preparation is well-suited for long term intra-oral deposition and mineral or drug release in the singular environment of the oral cavity.

24. The pharmaceutical preparation of claim 1, wherein the binder is at least one member selected from the group consisting of PVP, Methocel, Ethocel and Klucel.

25. The pharmaceutical preparation of claim 1, wherein the lubricant is at least one member selected from the group consisting of Syloid, PEG, Mg Stearate and Pruv.

26. The pharmaceutical preparation of claim 1, comprising a coating layer being a rate controlling polymer comprising at least one member selected from the group consisting of Ethocel (Ethylcellulose), Cellulose Acetate, Acrylic Polymers, HPMC (Hydroxypropyl Methylcellulose), Hydroxy Propyl Cellulose, and Polyox (high MW polyethylene oxide).

27. The pharmaceutical preparation of claim 1, wherein the coating layer is a semi permeable membrane and the dosage form comprises osmotic components.

28. The pharmaceutical preparation of claim 1, comprising a carrier base material that includes at least one member selected from the group consisting of hydrophilic polymers, hydroxypropylmethyl cellulose (HPMC), hydroxypropyl cellulose (HPC), GUMS, polyethylene oxide (Polyox), polyvinyl pyrolidone (PVP), hydrophobic polymers, ethyl cellulose (Ethocel), acrylate based polymers, Eudragit, PVA, and Kollidone SR.

29. The pharmaceutical preparation of claim 1, further comprising a filler selected from at least one of microcrystalline cellulose and lactose; and a flavor comprising at least one member selected from the group consisting of Menthol, Saccharin, and Vanillin.

30. A method for the preparation of a pharmaceutical preparation comprising preparing the controlled long acting release pharmaceutical preparation of claim 1.

31. A method for utilizing a pharmaceutical preparation comprising utilizing the controlled long acting release pharmaceutical preparation of claim 1.

32. A method comprising applying the pharmaceutical preparation of claim 1 for an extended term in an oral cavity.

33. A method for promoting tooth re-mineralization, for avoiding tooth de-mineralization, and for applying a long term anti-demineralization therapy, comprising utilizing the using a pharmaceutical preparation of claim 1.

34. A method of manufacturing a medicament for treatment of diseases in an oral cavity, comprising utilizing the pharmaceutical composition of claim 1.

35. A method of manufacturing a medicament for absorption of upper parts of a GI tract, comprising utilizing the pharmaceutical composition of claim 1.

36. A pharmaceutical preparation according to claim 1, being stored in a device located in an oral cavity for at least one of (a) the treatment of diseases of the oral cavity; or (b) releasing the pharmaceutical preparation into said oral cavity.

37. A device for maintaining a preparation according to claim 1, wherein said device has been in position in an oral cavity for a period of time in the range of from several hours to several months.

38. (canceled)

39. The pharmaceutical preparation of claim 6, further comprising pore forming agents.
